(19) United States
(12) Patent Application Publication
(10) Pub. No.: US 2025/0098970 A1
(43) Pub. Date: Mar. 27, 2025

(54) PHYSIOLOGICAL MONITOR WITH ELECTRICAL REFERENCE SYSTEM

(71) Applicant: Integra LifeSciences Switzerland Sàrl, Le Locle (CH)

(72) Inventors: Jason Lewinski, Hudson, MA (US); Salim Kassem, North Attleboro, MA (US); Quentin Aten, Knoxville, TN (US); Joseph Thomas Roberto, Brighton, MA (US)

(21) Appl. No.: 18/891,735

(22) Filed: Sep. 20, 2024

Related U.S. Application Data
(60) Provisional application No. 63/585,023, filed on Sep. 25, 2023.

(51) Int. Cl. A61B 5/03; A61B 5/00

(52) U.S. Cl. CPC A61B 5/031; A61B 5/6833; A61B 5/6868; A61B 5/742; A61B 2562/0247; A61B 2562/225

(57) ABSTRACT

A pressure monitor device includes a console having a display monitor and a plurality of input and output ports. An extension cable includes a distal end having a connector that connects to at least one of the plurality of input and output ports formed on the console, and a proximal end having a junction. A sensor mechanism includes a sensor mechanism reference cable for coupling the sensor mechanism to the junction of the extension cable. An electrical reference system includes a reference electrode and a patient reference cable having a proximal end communicatively coupled to the extension cable and a distal end communicatively coupled to the reference electrode. The reference electrode establishes a reference electrical potential of an external environment to electrically equilibrate the pressure monitor device.

### INCORPORATION BY REFERENCE

Applications for which a foreign or domestic priority claim are identified in the Application Data Sheet as filed with the present application are hereby incorporated by reference herein and made a part of the present disclosure.

### TECHNICAL FIELD

The present disclosure generally relates to medical devices, and more particularly, to physiological parameter monitors, such as pressure monitors, equipped with an electrical referencing system for enhanced measurement accuracy.

### BACKGROUND

Traditional monitors are utilized to measure physiological parameters within the body. However, these devices may be susceptible to electrical interference from an external environment that may lead to incorrect readings. In some instances, these inaccurate readings may influence medical treatment decisions. Accordingly, a need exists for a monitor device that is capable of minimizing and/or eliminating electrical interference from an external environment in order to ensure more accurate readings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 depicts a schematic view of a pressure monitor device, according to one or more embodiments shown and described herein;

FIG. 2 depicts a schematic view of an electrical reference system of the pressure monitor device of FIG. 1, according to one or more embodiments shown and described herein;

FIG. 3 depicts a schematic view of another embodiment of an electrical reference system of the pressure monitor device of FIG. 1, according to one or more embodiments shown and described herein;

FIG. 4 depicts a schematic view of another embodiment of an electrical reference system of the pressure monitor device of FIG. 1, according to one or more embodiments shown and described herein;

FIG. 5 depicts a schematic view of another embodiment of an electrical reference system of the pressure monitor device of FIG. 1 disposed in a target area, according to one or more embodiments shown and described herein;

FIG. 6 depicts a schematic view of a sensor of the electrical reference system of FIG. 5, according to one or more embodiments shown and described herein; and

FIG. 7 depicts an illustrative method of performing pressure monitoring, according to one or more embodiments shown and described herein.

### DETAILED DESCRIPTION

Embodiments disclosed herein relate to pressure monitor devices and electrical reference systems for pressure monitors. More specifically, the present disclosure relates to a pressure monitor device that includes a sensing mechanism, a display monitor, an extension cable electrically coupled to the sensing mechanism and the display monitor, and an electrical reference system. In these embodiments, the electrical reference system may include a patient reference cable that is electrically and/or communicatively coupled to the extension cable, and a reference electrode that is electrically and/or communicatively coupled to the patient reference cable. The reference electrode of the electrical reference system may reference the electrical potential of the environment of the sensing mechanism relative to a lowest electrical potential of the sensor mechanism in order to equilibrate any bias voltage, thereby providing a more accurate sensor measurement.

In other embodiments, the electrical reference system may be embedded into the sensing mechanism in order to minimize connections within the monitoring device. For example, in these embodiments, the electrical reference system may include a reference electrode that is integrated into the sensing mechanism, which may effectively minimize the distance (and potential interference) between the sensing mechanism and the electrical reference system.

As has been noted herein, intracranial pressure (“ICP”) monitors are devices used to measure the pressure inside the skull. This pressure may increase due to various medical conditions such, as traumatic brain injury, hydrocephalus, tumors, and other types of brain related conditions. In some instances, elevated ICP may be harmful, as the increased pressure within the skull may cause brain tissue to be compressed, thereby reducing blood flow to the brain. The ICP monitor devices described herein may help alleviate risks associated with elevated ICP by continuously monitoring and displaying ICP to medical personnel, such that timely action may be taken in the event ICP levels are elevated to and/or maintained at a level that requires medical intervention.

In operation, traditional ICP monitor devices may include a sensor that is implanted into the brain and connected (e.g., via wire or any other similarly communicative coupling) to an external monitor of the device to display ICP readings that are used to guide patient treatment. However, the sensors utilized in many ICP monitor devices are inherently susceptible to environmental noise (or any other voltage potential difference between the sensor and the environment), which can influence the accuracy of the sensor reading. This electrical interference from the external environment can, in some instances, result in incorrect pressure readings being monitored and displayed in some ICP monitor devices, which may have an impact on treatment plans for patients.

To minimize inaccurate readings, the disclosed ICP monitor device utilizes an electrical reference connection between the monitoring system and the patient to equilibrate the electrical potential the sensor mechanism is exposed to For example, it should be appreciated that any environment has an inherent electrical potential that may be influenced by nearby electrical devices, electromagnetic fields, and/or physiological processes in the body. By equilibrating the electrical potential internal to the sensor with the external environment (i.e., patient), the ICP monitor device is provided a consistent reference against which to measure ICP. Accordingly, the electrical environment where the sensor operates is considered common mode (i.e., the same all around) and will therefore not affect the performance of the device.

Embodiments of pressure monitor devices and electrical reference systems will now be described in more detail herein. The following will now describe these devices and systems with reference to the drawings and where like numbers refer to like structures.

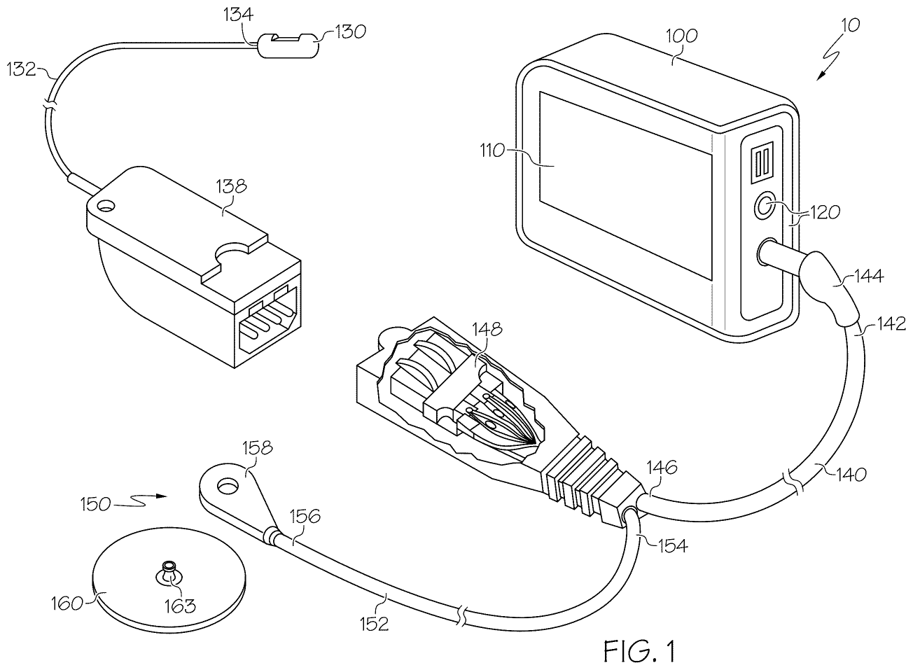

Referring now to FIG. 1, a schematic view of a pressure monitor device 10, such as an ICP monitor device, is depicted. The ICP monitor device 10 may include a console 100 having a monitor 110, such as a display monitor for visually and/or audibly displaying ICP readings, and a plurality of input and output ports 120 for coupling a sensor mechanism 130, such as an ICP sensor, and an electrical reference system 150 to the console 100, as will be described in additional detail herein. In these embodiments, the ICP monitor device 10 may further include at least one extension cable 140, which may be used to electrically and/or communicatively couple the sensor mechanism 130 and the electrical reference system 150 to the plurality of input and output ports 120 of the console 100.

Although the ICP monitor device 10 depicted in FIG. 1 is illustrated as including the plurality of input and output ports 120 and the at least one extension cable 140, it should be appreciated that, in some embodiments, the sensor mechanism 130 may be wirelessly coupled to the console 100. For example, in these embodiments, the sensor mechanism 130 may be coupled to the console using telemetry (e.g., providing a transmitter in the sensor mechanism 130 that transmits data to a receiver positioned in the console 100), Bluetooth, Wi-Fi, or any other similar wireless coupling. It should be noted that, in embodiments in which the sensor mechanism 130 is wirelessly coupled to the console 100, the console 100 may further include mechanisms for ensuring the synchronization of the sensor mechanism 130 ensure that latency and/or misalignment in data transmission does not affect the accuracy of the ICP readings obtained by the ICP monitor device 10.

In the embodiments described herein, the ICP monitor device 10 may further include a power source (not depicted) for supplying power to the ICP monitor device 10. For example, in some embodiments, the ICP monitor device 10 may include a battery-powered power source, which may allow the ICP monitor device 10 to be utilized during patient transport (e.g., in an ambulance or otherwise). In other embodiments, the power source may include an external power source that is configured to be powered via a standard wall outlet or any other similar power source.

Referring still to FIG. 1, the extension cable 140 may include a distal end 142 having a connector 144 for coupling to the console 100 and a proximal end 146 including a junction 148 for coupling to the sensor mechanism 130 and/or the electrical reference system 150. In these embodiments, the extension cable 140 may be formed of copper, tinned copper, silver or silver-plated copper, gold or gold-plated copper, or any other material capable of effectively transmitting signals from the sensor mechanism 130 and/or the electrical reference system 150 to the console 100. Furthermore, the extension cable 140 may be insulated with polyethylene, polytetrafluoroethylene (PTFE), silicone, santoprene, or any other similar elastomer having sufficient flexibility and biocompatibility.

As further depicted in FIG. 1, the sensor mechanism 130 may be coupled to the extension cable 140 via a sensor mechanism reference cable 132. In these embodiments, the sensor mechanism reference cable 132 may include a proximal end 134 that is integrated into and/or coupled to the sensor mechanism 130, and a distal end 136 having an adapter 138 for coupling the sensor mechanism 130 to the junction 148 of the extension cable 140. It should be appreciated that, in the embodiments described herein, the adapter 138 may be any adapter capable of facilitating the transmittal of ICP signals from the sensor mechanism 130 to the console 100.

It should be further noted that the sensor mechanism 130 may be any sensor capable of accurately recording ICP values and relaying ICP values to the console 100 in real-time. For example, in the embodiments described herein, the sensor mechanism 130 may include a strain gauge transducer (e.g., microtransducer), a piezoelectric sensor, a capacitive sensor, or any other similar sensor mechanism. In these embodiments, it should be appreciated that the sensor mechanism 130 utilized in the ICP monitor device 10 may depend on a variety of factors, including accuracy, sensitivity, size constraints, and the environment in which the ICP monitor device 10 is utilized (e.g., operating room, MRI, etc.).

Referring now to FIGS. 1-4, embodiments of the electrical reference system 150 are depicted. As discussed herein, it should be appreciated that the electrical reference system 150 is configured to provide a stable and/or known reference point against which other electrical measurements (e.g., ICP readings) may be compared.

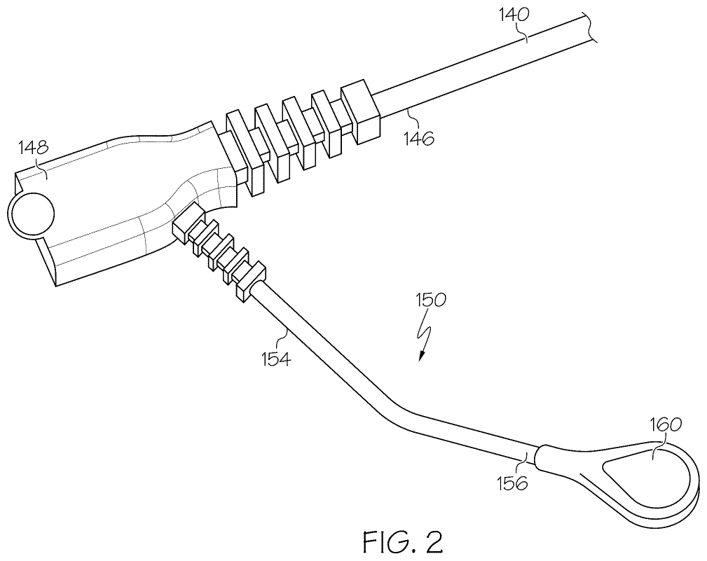

As depicted in FIGS. 1-4, the electrical reference system 150 may include a patient reference cable 152 having a proximal end 154 that is electrically and/or communicatively coupled to the extension cable 140, and a distal end 156 that is coupled to a reference electrode 160. For example, as depicted in FIGS. 1 and 2, the proximal end 154 of the patient reference cable 152 may be integrally formed with the junction 148 of the extension cable 140, while the distal end 156 includes an electrode connector 158 that is configured to couple (e.g., releasably or otherwise) the patient reference cable 152 to the reference electrode 160. In these embodiments, the patient reference cable 152 may be a standard electrocardiogram (“ECG”) cable, such as a tinned copper wire with a santoprene insulation coating, and may be integrally formed with and/or fixedly coupled to the junction 148 of the extension cable 140, such as by soldering, welding, overmolding, or any other similar method. By fixedly coupling the proximal end 154 of the patient reference cable 152 to the junction 148 of the extension cable 140, it may be possible to ensure the physical integrity and reliability of the connection between the patient reference cable 152 and the extension cable 140.

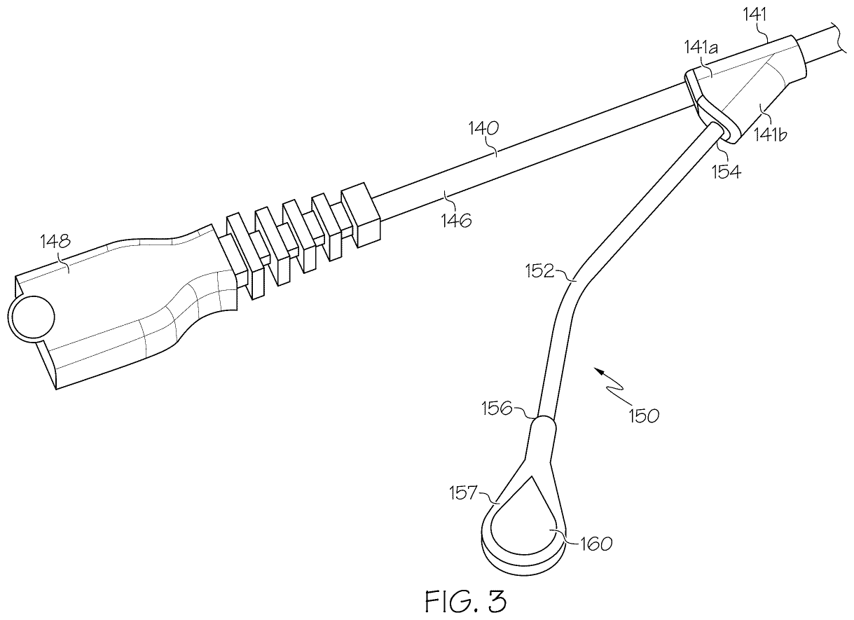

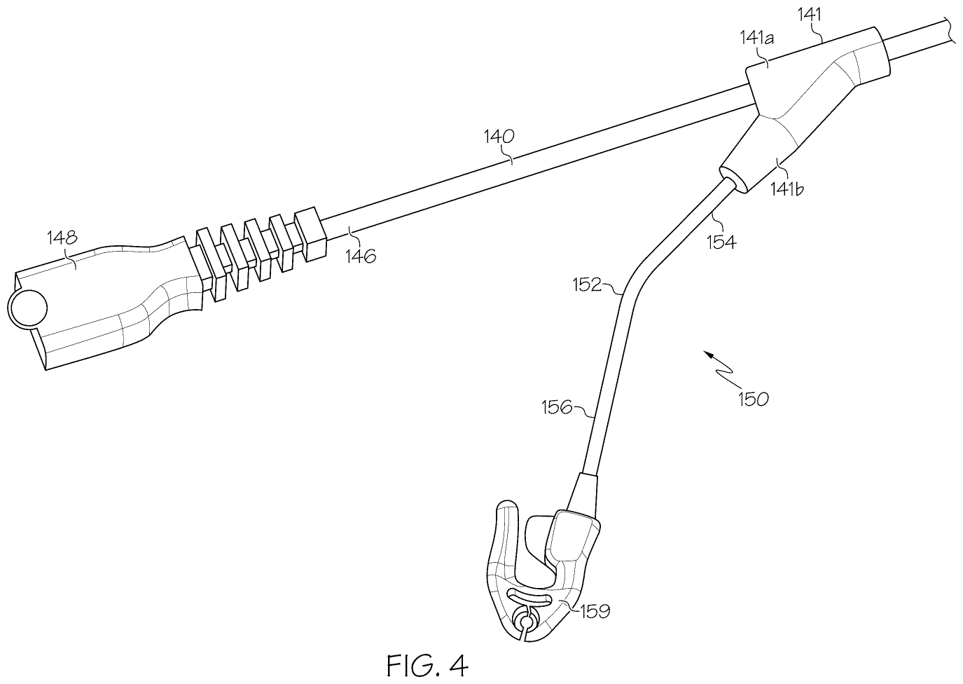

While the patient reference cable 152 may be integrally formed with the junction 148 of the extension cable 140, in some embodiments, the extension cable 140 may further include a y-connector 141, such as an electrical y-connector, which may be used to couple the patient reference cable 152 to the extension cable 140. For example, as depicted in FIGS. 3 and 4, the y-connector 141 may include a first port 141a configured for connecting to the sensor mechanism 130 and a second port 141b configured for connecting to the patient reference cable 152. In these embodiments, the patient reference cable 152 may be integrally formed with and/or electrically and/or communicatively coupled to the second port 141b. In these embodiments, the proximal end 154 of the patient reference cable 152 may be overmolded to the second port 141b of the y-connector 141 in order to ensure the physical integrity and reliability of the connection, as has been described herein.

Although the patient reference cable 152 is illustrated as being a standard ECG cable, it should be appreciated that, in the embodiments described herein, the patient reference cable 152 may include any cable and/or materials capable of transmitting electrical signals obtained from the reference electrode 160 to the console 100.

Referring again to FIGS. 1-4, the reference electrode 160 may be coupled to the distal end 156 of the patient reference cable 152 such that electrical signals obtained by the reference electrode 160 may be transmitted via the patient reference cable to the console 100. For example, as depicted in FIGS. 2 and 3, the reference electrode 160 may be integrally formed with the distal end 156 of the patient reference cable 152. In these embodiments, the distal end 156 of the patient reference cable 152 may include an overmold portion 157, which may be disposed at least partially around the reference electrode 160 and ensure that a reliable electrical coupling is achieved between the reference electrode 160 and the patient reference cable 152. Furthermore, in the embodiments described herein, the reference electrode 160 may be a skin patch electrode, or any other similar electrode that may be adhered or otherwise removably attached to a patient to establish an electrical potential of the patient, as will be described in additional detail herein.

In other embodiments, the distal end 156 of the patient reference cable 152 may include a coupler 159, such as a clip, clamp, or other similar releasable coupling, which is configured to connect with a receiver 163 formed on the reference electrode 160. For example as depicted in FIGS. 1 and 4, the coupler 159 may be secured to the receiver 163 of the reference electrode 160 to establish an electrical connection between the reference electrode 160 and the patient reference cable 152.

Operation of the ICP monitor device 10 will now be described in detail with reference to FIGS. 1-6. In the embodiments described herein, an operator may conduct an ICP monitoring procedure by connecting the various components of the ICP monitor device 10 to the console 100. For example, the extension cable 140 may be coupled to the electrical reference system 150 and the sensor mechanism 130, and the console 100 via the plurality of input and output ports 120. With the extension cable 140 paired with the console 100, the reference electrode 160 may be adhered to a patient, and the patient reference cable 152 may be secured to the reference electrode 160.

Once the reference electrode 160 is adhered to the patient, the reference electrode 160 may monitor the patient's electrical potential, which may be used as a reference electrical potential when monitoring ICP readings in a target area (e.g., brain). The implementation of the reference electrical potential equilibrates the environment in which the sensor operates, thereby nullifying potential bias from the external environment. More specifically, in the embodiments described herein, the reference electrical potential may be used to convert AC noise and DC offset voltage bias into common mode signals, which may lead to more accurate readings.

It should be further noted that the removal of the DC offset bias may further aid in preventing ion migration within the implanted sensor. As provided herein, ion migration may refer to the movement of charged particles (e.g., ions) across a medium due to a potential difference. In these embodiments, the presence of DC voltage bias may drive movement of electrolytes from a patient's brain towards the sensor mechanism 130. This migration can interfere with the sensing mechanism and result in inaccurate readings.

With the reference electrical potential established via the reference electrode 160, the sensor mechanism 130 may be positioned within the target area, such that the sensor mechanism 130 monitors ICP levels within the target area. The ICP levels monitored by the sensor mechanism 130 may be relayed as electrical signals from the sensor mechanism 130 to the console 100 via the extension cable 140, with the console 100 being configured to convert the electrical signals from the sensor mechanism 130 into a graphical, visual, and/or audible display provided on the monitor 110 of the console 100. For example, the electrical signals representing the ICP readings obtained by the sensor mechanism 130 may be displayed as histograms or other graphical representations via the monitor 110.

Referring still to FIGS. 1-4, the console 100 may be configured to ensure that the ICP levels within the target area remain within a predetermined range, which may be programmed into the console 100 or established via user input on the console 100. In these embodiments, the console 100 may include a plurality of alarms configured to indicate to a user when an ICP level falls outside of the predetermined range. For example, the plurality of alarms may provide audible and/or visual feedback to indicate to the user that the ICP level falls outside of the predetermined range. Accordingly, in the event that ICP levels exceed a predetermined upper threshold, or fall below a predetermined lower threshold, the console 100 may trigger at least one of the plurality of alarms to provide a warning to the user.

In some embodiments, the console 100 may be further configured to trigger at least one of the plurality of alarms when the monitored ICP levels are sustained outside the predetermined range for a predetermined period of time. For example, in these embodiments, the console 100 may not immediately trigger at least one of the plurality of alarms when the ICP levels monitored by the sensor mechanism 130 fall outside the predetermined range. Instead, the console 100 may further track the amount of time the ICP levels remain outside of the predetermined range, such that at least one of the plurality of alarms is only triggered when the ICP levels remain outside the predetermined range for a time period that exceeds the predetermined period of time.

Referring still to FIGS. 1-4, in the embodiments described herein, the console 100 may further include a plurality of memory and data components (not depicted) configured to store and/or save ICP data captured by the sensor mechanism 130. In these embodiments, the memory and data components may allow a technician or other user to monitor ICP data for certain trends that may be beneficial in developing treatment strategies for a particular patient.

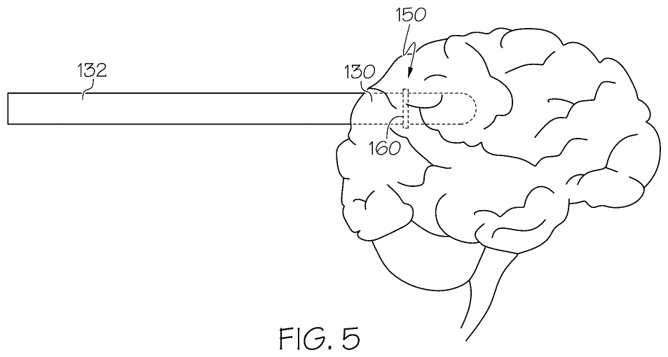

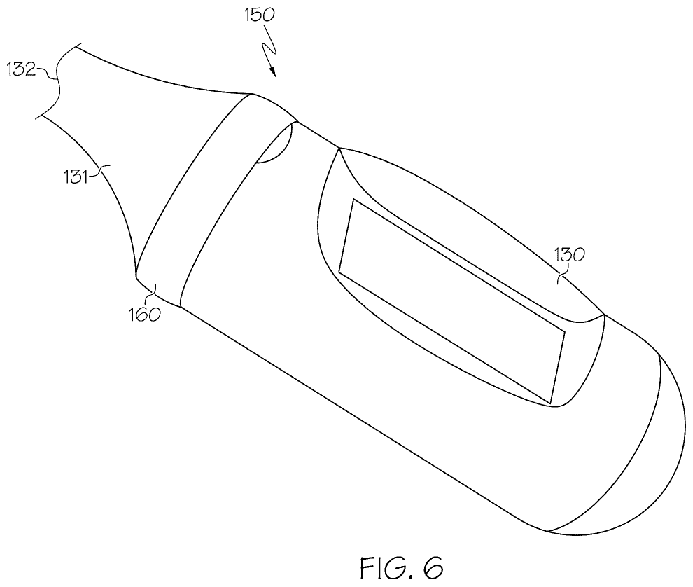

Turning now to FIGS. 5 and 6, another embodiment of an electrical reference system 150 for use in connection with the ICP monitor device 10 is disclosed. In these embodiments, the reference electrode 160 of the electrical reference system 150 may be directly disposed on the sensor mechanism 130. For example, as depicted in FIGS. 5 and 6, the reference electrode 160 may be an electrode ring that may be embedded or otherwise integrated into the sensor mechanism 130. Although the reference electrode 160 is depicted as being an electrode ring, it should be appreciated that the reference electrode 160 may take any shape, such as a strip disposed on a side of the sensor mechanism 130, without departing from the scope of the present disclosure.

As most clearly depicted in FIG. 6, the reference electrode 160 may be disposed about a first end 131 of the sensor mechanism (e.g., the end of the sensor mechanism 130 adjacent the distal end 136 of the sensor mechanism reference cable 132), such that an electrical and/or communicative coupling may be formed between the reference electrode 160 and the sensor mechanism reference cable 132. Although the reference electrode 160 is depicted as being disposed (e.g., embedded or otherwise) about the first end 131 of the sensor mechanism 130, it should be appreciated that the reference electrode 160 may be disposed on any portion of the sensor mechanism 130 without departing from the scope of the present disclosure.

In the embodiments described herein, the reference electrode 160 may identify the electric potential of the patient as soon as the sensor mechanism 130 (and, in turn the reference electrode 160) contacts tissue positioned at the target area (e.g., brain), which provides an immediate and direct referencing of the patient's electrical environment. As has been described in detail herein, utilizing the electric potential of the patient as a reference point eliminates potential offset bias that would be present without a reference between the patient and the sensor mechanism 130 and the monitoring device 100. Furthermore, it should be appreciated that the proximity of the reference electrode 160 to the sensor mechanism 130 may further aid in minimizing potential bias that may interfere with the sensor mechanism 130.

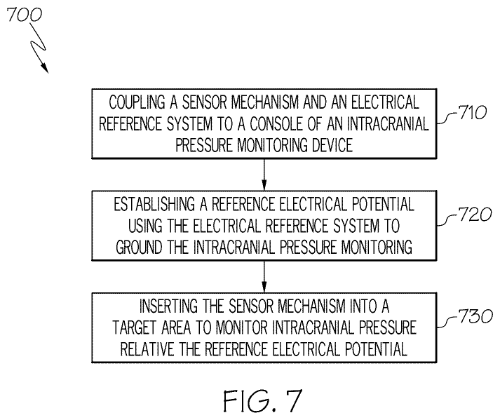

Referring now to FIG. 7, an illustrative method 700 of performing pressure monitoring is depicted. In these embodiments, the method 700 may first involve coupling a sensor mechanism and an electrical reference system to a console of an ICP monitoring device using an extension cable, as shown at block 710. In some embodiments, the extension cable may further include a y-connector with a first port for coupling the extension cable to the sensor mechanism and a second port for coupling the extension cable to the electrical reference system. In other embodiments, the senor mechanism and/or the electrical reference system may be integrated into (e.g., overmolded or otherwise) the extension cable, as has been described herein.

With the sensor mechanism and the electrical reference system communicatively coupled to the console, the method may advance to block 720, which may involve establishing a reference electrical potential using the electrical reference system. In these embodiments, establishing the reference electrical potential may involve adhering (or otherwise securing) a reference catheter to an external environment, such as a patient's skin. Once the reference electrical potential is established, the sensor mechanism may be inserted into a target area, such as a patient's brain, to monitor pressure relative the reference electrical potential, as shown at block 730.

As should be appreciated in view of the foregoing, a pressure monitor device is disclosed herein. The ICP monitor device may include a sensor mechanism communicatively coupled to a monitor display of a console of the device via an extension cable. The ICP monitor device may further include an electrical reference system having a reference electrode and a patient reference cable that extends between and communicatively couples the reference electrode to the extension cable, and in turn, the monitor display of the console. In the embodiments described herein, the reference electrode may be attached to a patient, such that the reference electrode may utilize the patient's electrical potential as a reference point. As described herein, utilizing the electrical potential of the patient as a reference point may effectively equilibrate the ICP monitor device to the patient, thereby nullifying potential bias from external electrical interferences.

In addition to ICP monitor devices illustrated herein, the embodiments shown and described herein can apply to monitoring devices for other patient physiological parameters, such as oxygen saturation in the brain or other tissue, temperature in the brain or other tissue or organ, heart rate, flow rate through an implantable valve, fluid pressure in an implantable device, blood pressure, cerebral perfusion pressure, pressure reactivity index, pressure-volume compensatory reserve index, and gastric pH level. The electrical reference systems described herein can be applied to a variety of sensors measuring various physiological parameters, including but not limited to, pressure, and can be used with pressure sensors measuring various types of pressure at different physiological locations in the patient.

The terminology used herein is for the purpose of describing particular aspects only and is not intended to be limiting. As used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

What is claimed is:

1. A pressure monitor device comprising:
a console including a display monitor and a plurality of input and output ports;
an extension cable including a distal end having a connector that connects to at least one of the plurality of input and output ports formed on the console, and a proximal end having a junction;
a sensor mechanism including a sensor mechanism reference cable for coupling the sensor mechanism to the junction of the extension cable; and
an electrical reference system comprising:
a reference electrode; and
a patient reference cable having a proximal end communicatively coupled to the extension cable and a distal end communicatively coupled to the reference electrode;
wherein the reference electrode is configured to establish a reference electrical potential of an external environment to electrically equilibrate the pressure monitor device.

2. The pressure monitor device of claim 1, further comprising a battery-powered power source.

3. The pressure monitor device of claim 1, wherein a distal end of the sensor mechanism reference cable includes an adapter for coupling the sensor mechanism to the junction of the extension cable.

4. The pressure monitor device of claim 1, wherein the sensor mechanism is configured to monitor pressure readings in target area.

5. The pressure monitor device of claim 1, wherein the proximal end of the patient reference cable is overmolded to the junction of the extension cable.

6. The pressure monitor device of claim 1, wherein the extension cable further include a y-connector having a first port and a second port, the first port being configured to receive the sensor mechanism reference cable and the second port being configured to receive the proximal end of the patient reference cable.

7. The pressure monitor device of claim 1, wherein the patient reference cable is a tinned copper wire cable with a santoprene insulation coating.

8. The pressure monitor device of claim 1, wherein the reference electrode is integrally formed with the distal end of the patient reference cable.

9. The pressure monitor device of claim 1, wherein the distal end of the patient reference cable include an overmold portion that is disposed at least partially around the reference electrode.

10. The pressure monitor device of claim 1, wherein the distal end of the patient reference cable includes a coupler configured to connect with a receiver formed on the reference electrode.

11. The pressure monitor device of claim 1, wherein the reference electrode is a skin-patch electrode.

12. The pressure monitor device of claim 1, further including a plurality of alarms that are triggered when pressure values monitored by the sensor mechanism fall outside of a predetermined range.

13. The pressure monitor device of claim 1, further including a plurality of alarms that are triggered when pressure values monitored by the sensor mechanism fall outside of a predetermined range and remain outside of the predetermined range for a predetermined period of time.

14. The pressure monitor device of claim 1, further comprising a plurality of memory components configured to save data captured by the sensor mechanism.

15. A pressure monitor device comprising:
a console including a display monitor and a plurality of input and output ports;
an extension cable including a distal end having a connector that connects to at least one of the plurality of input and output ports formed on the console, and a proximal end having a junction;
a sensor mechanism including a sensor mechanism reference cable for coupling the sensor mechanism to the junction of the extension cable; and
an electrical reference system including a reference electrode disposed on the sensor mechanism;
wherein the reference electrode is configured to establish a reference electrical potential of an external environment to electrically equilibrate the pressure monitor device.

16. The pressure monitor device of claim 15, wherein the reference electrode is an electrode ring.

17. The pressure monitor device of claim 15, wherein the reference electrode is embedded in a first end of the sensor mechanism adjacent the distal end of the sensor mechanism reference cable.

18. The pressure monitor device of claim 15, wherein the reference electrode is communicatively coupled to the sensor mechanism reference cable.

19. A method of monitoring pressure comprising:
coupling a sensor mechanism and an electrical reference system to a console of a pressure monitoring device using an extension cable;
establishing a reference electrical potential of an external environment using the electrical reference system;
grounding, using the reference electrical potential, the pressure monitoring device; and
inserting the sensor mechanism into a target area;
monitoring, using the sensor mechanism, pressure relative the reference electrical potential.

20. The method of claim 19, wherein establishing the reference electrical potential further comprises adhering a reference electrode of the electrical reference system to the external environment.
